(19) United States
(12) Patent Application Publication
(10) Pub. No.: US 2004/0053264 A1
(43) Pub. Date: Mar. 18, 2004

(54) Clinical panel assay using DNA chips

(76) Inventor: Sung Sup Park, Seoul (KR)

Correspondence Address: KNOBBE MARTENS OLSON & BEAR LLP, 2040 MAIN STREET, FOURTEENTH FLOOR, IRVINE, CA, 92614, US

(21) Appl. No.: 10/357,306

(22) Filed: Jan. 30, 2003

Related U.S. Application Data
(60) Provisional application No. 60/354,079, filed on Feb. 1, 2002.

(51) Int. Cl. C12Q 1/68; C12M 1/34

(52) U.S. Cl. 435/6; 435/287.2

(57) ABSTRACT

The invention relates to a clinical panel, a microarray (e.g., a DNA chip), and a method, for determining a causative agent of human disease, more particularly, infectious disease and mitochondrial disorders. The clinical panel of the invention contains at least one pair of oligonucleotides (reverse primers, forward primers), whereby both oligonucleotides of the pair are suitable for use as primers for the amplification by means of polymerase chain reaction of one or both complementary strands of a DNA segment of a human infectious agent or human mitochondria DNA containing a mutation.

### BACKGROUND OF THE INVENTION

1. Field of the Invention

The present invention relates to a clinical panel for differential diagnosis of human disease, more specifically, to a clinical panel for screening of clinical samples for human pathogens and genetic disorders using a DNA chip, and a method for differential diagnosis of human disease using said clinical panel.

2. Description of the Related Art

The classical method for detecting a pathogen (bacteria, yeast or virus) in a clinical sample involves culturing of the sample in order to expand the number of pathogen present into observable colony growths which can be identified and enumerated. If desired, the cultures can also be subjected to additional testing in order to determine susceptibility of a pathogen to drug treatment. For accurate identification of the infecting species the clinician must rely on culture results which can require anywhere from 3 days to 8 weeks of growth followed by extensive biochemical testing that may require additional days or even weeks of tests, while it is often important to make this determination quickly due to the severity of the disease and thus the necessity of immediate drug intervention.

Mitochondrial (mt) DNA mutations have been linked to seizures, strokes, optic atrophy, neuropathy, myopathy, cardiomyopathy, sensorineural hearing loss, diabetes mellitus, and other clinical features. Mitochondrial DNA mutations also may play an important role in aging, as well as in common age-related neurodegenerative disorders such as Parkinson's disease. Therefore, it is becoming increasingly important for clinicians to recognize the clinical syndromes suggestive of a mitochondrial disorder, and to understand the unique features of mitochondrial genetics that complicate diagnosis and genetic counseling. The wide variety of clinical manifestations associated with mt disorders highlights the importance of understanding the principles of mt genetics as they relate to human diseases.

Genetic tests are commercially available for the most common mutations associated with some of the mt syndromes. Several research laboratories can assist with evaluation for more rare mtDNA mutations. However, a fast and comprehensive testing of a wide range of mt mutations is needed for a definite diagnosis of a mt genetic disorder which is important in genetic counseling as well as in choosing correct and timely treatment options.

In the field of molecular biology, microarray chips, as represented by biochip on which biopolymers such as DNAs or proteins are immobilized, have lately become of interest. For an experiment using microarray chip, numbers of supports (chips) with diverse types of elements (probes) immobilized thereon are produced. The elements on each chip are subjected to reaction with a sample (target) of a test subject. The difference of the reaction between multiple elements and the sample on one chip may be detected by a single experiment.

### SUMMARY OF THE INVENTION

We describe herein a novel method and means to diagnose human disease by designing a novel clinical panel comprising a DNA chip for simultaneous detection in a clinical sample of any group of pathogenic organisms or mitochondrial mutations causing diseases with similar symptoms.

As described below, the present invention has significant advantages over prior art methods not only in the enhanced accuracy, specificity and simplicity of the test, but also in greatly reducing the time to achieve a diagnosis. The invention makes possible a definitive diagnosis and initiation of effective treatment on the same day as testing.

### BRIEF DESCRIPTION OF THE DRAWINGS

FIG. 1 is the schematic diagram of the human mitochondrial genome, indicating the approximate locations and nucleotide positions of the mtDNA mutations most commonly associated with selected mitochondrial disorders. Solid lines between the circles (representing the two strands of mtDNA) represent the borders of tRNA gene locations. See text for explanation of abbreviations of mitochondrial disorders. Protein-encoding genes are labeled as follows: ND1-6 and ND4L are subunits of NADH ubiquinone oxidoreductase (complex I); COX I-III are cytochrome oxidase (complex IV) subunits; A8 and A6 are ATPase (complex V) subunits; cyt b represents the “cytochrome b” subunit of complex III.

### DETAILED DESCRIPTION OF THE PREFERRED EMBODIMENT

The clinical panel of the invention for differential diagnosis of human disease comprises: a DNA chip with probes that have nucleotide sequences complementary to DNA of pathogens or to human mitochondrial DNA with known or suspected mutations; primers for amplifying DNA obtained from clinical samples; and means for labeling amplified sample DNA hybridized with the probes said DNA chip. The DNA chip may further comprise position markers to locate probes, and staining or labeling is performed by using means for labeling.

For clarity of disclosure, and not by way of limitation, the detailed description of the invention is divided into the subsections that follow.

### A. Definitions

Unless defined otherwise, all technical and scientific terms used herein have the same meaning as is commonly understood by one of ordinary skill in the art to which this invention pertains. All patents, applications, published applications and other publications referred to herein are incorporated by reference in their entirety. If a definition set forth in this section is contrary to or otherwise inconsistent with a definition set forth in applications, published applications and other publications that are herein incorporated by reference, the definition set forth in this section prevails over the definition that is incorporated herein by reference.

As used herein, “microarray chip”, “DNA chip”, “gene chip”, “biochip” refers to a solid substrate with a plurality of one-, two- or three-dimensional micro structures or micro-scale structures on which certain processes, such as physical, chemical, biological, biophysical or biochemical processes, etc., can be carried out. The microstructures or micro-scale structures such as, channels and wells, are incorporated into, fabricated on or otherwise attached to the substrate for facilitating physical, biophysical, biological, biochemical, chemical reactions or processes on the chip. The chip may be thin in one dimension and may have various shapes in other dimensions, for example, a rectangle, a circle, an ellipse, or other irregular shapes. The size of the major surface of chips can vary considerably, e.g., from about 1 mm² to about 0.25 m². Preferably, the size of the chips is from about 4 mm² to about 25 cm² with a characteristic dimension from about 1 mm to about 5 cm. The chip surfaces may be flat, or not flat. The chips with non-flat surfaces may include channels or wells fabricated on the surfaces.

As used herein, “moiety” encompasses both test moiety and target moiety. Non-limiting examples of moieties include cells, cellular organelles, viruses, particles, molecules, e.g., proteins, DNAs and RNAs, or an aggregate or complex thereof.

As used herein, “bacteria” refers to small prokaryotic organisms (linear dimensions of around 1 micron) with non-compartmentalized circular DNA and ribosomes of about, 70S.

As used herein, “virus” refers to an obligate intracellular parasite of living but non-cellular nature, consisting of DNA or RNA and a protein coat. Viruses range in diameter from about 20 to about 300 mn. Class I viruses (Baltimore classification) have a double-stranded DNA as their genome; Class II viruses have a single-stranded DNA as their genome; Class III viruses have a double-stranded RNA as their genome; Class IV viruses have a positive single-stranded RNA as their genome, the genome itself acting as mRNA; Class V viruses have a negative single-stranded RNA as their genome used as a template for mRNA synthesis; and Class VI viruses have a positive single-stranded RNA genome but with a DNA intermediate not only in replication but also in mRNA synthesis. The majority of viruses are recognized by the diseases they cause.

As used herein, “fungus” refers to a morphologically and physiologically diverse group of simple eukaryotic organisms. Some fungi grow as single cells (yeasts), others as multinuclear filaments (molds) and still others can grow as either single cells or multicellular filaments (dimorphic fungi).

As used herein, “mitochondria” refers to membrane-bounded organelles of the cell where oxidative phosphorylation takes place to generate ATP. Oxidative phosphorylation involves the transfer of electrons to oxygen coupled to the synthesis of ATP.

As used herein, “sample” refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid, ear discharge, or the like. Biological tissues are aggregates of cells, usually of a particular kind together with their intercellular substance that form one of the structural materials of a human body, including connective, epithelium, muscle and nerve tissues. Examples of biological tissues also include organs, tumors, lymph nodes, arteries and individual cell(s).

As used herein, “nucleotide” refers to a subunit of a nucleic acid consisting of a phosphate group, a 5′ carbon sugar and a nitrogen-containing base. In RNA the 5′ carbon sugar is ribose. In DNA, it is a 2-deoxyribose. The term also includes analogs of such subunits.

As used herein, “nucleotide polymer” refers to at least two nucleotides linked by phosphodiester bonds.

As used herein, “oligonucleotide” refers to a nucleotide polymer generally about 10 to about 100 nucleotides in length, but which may be greater than 100 nucleotides in length.

As used herein, “nucleic acid probe” refers to a single-stranded nucleic acid sequence that will combine with a complementary single stranded target nucleic acid sequence to form a double-stranded molecule (hybrid). A nucleic acid probe may be an oligonucleotide or a nucleotide polymer.

As used herein, “hybrid” refers to the complex formed between two single-stranded nucleic acid sequences by Watson-crick base pairings or non-canonical base pairings between the complementary bases.

As used herein, “hybridization” refers to the process by which two complementary strands of nucleic acids combine to form double stranded molecules (hybrids).

As used herein, “complementarity” refers to a property conferred by the base sequence of a single strand of DNA or RNA which may form a hybrid or double stranded DNA:DNA, RNA:RNA or DNA:RNA through hydrogen bonding between Watson-Crick base pairs on the respective strands. Adenine (A) usually complements thymine (T) or uracil (U), while guanine (G) usually complements cytosine (C).

As used herein, “stringency” refers to term used to describe the temperature and solvent composition existing during hybridization and the subsequent processing steps. Under high stringency conditions only highly homologous nucleic acid hybrids will form; hybrids without a sufficient degree of complementarity will not form. Accordingly, the stringency of the assay conditions determines the amount of complementarity needed between two nucleic acid strands forming a hybrid. Stringency is chosen to maximize the difference in stability between the hybrid formed with the target and the nontarget nucleic acid. Stringency of hybridization in determining percentage mismatch is as follows:

1) high stringency: 0.1×SSPE, 0.1% SDS, 65° C.;

2) medium stringency: 0.2×SSPE, 0.1% SDS, 50° C. (also referred to as moderate stringency); and

3) low stringency: 1.0×SSPE, 0.1% SDS, 50° C.

It is understood that equivalent stringencies may be achieved using alternative buffers, salts and temperatures.

As used herein, “probe specificity” refers to characteristic of a probe which describes its ability to distinguish between target and non-target sequences. Dependent on sequence and assay conditions, probe specificity may be absolute (i.e., probe able to distinguish between target organisms and any nontarget organisms), or it may be functional (i.e., probe able to distinguish between the target organism and any other organism normally present in a particular sample). Many probe sequences can be used for either broad or narrow specificity depending on the conditions of use.

As used herein, “gene” refers to the unit of inheritance that occupies a specific locus on a chromosome, the existence of which can be confirmed by the occurrence of different allelic forms. Given the occurrence of split genes, gene also encompasses the set of DNA sequences (exons) that are required to produce a single polypeptide.

As used herein, “DNA chip” refers to an array of oligonucleotides immobilized on a surface that can be used to screen an RNA sample (after reverse transcription) or DNA sample and thus a method for rapidly determining which pathogens or mutations are present in the clinical sample the RNA or DNA came from.

As used herein, “differential diagnosis” refers to determination of which two or more diseases with similar symptoms is the one from which a patient is suffering from based on an analysis of clinical data.

### B. Diseases and Pathogens

A number of human diseases caused by a variety of pathogens may clinically present themselves with similar symptoms, but require different treatment strategies and regimens.

### Meningitis

Meningitis is an inflammation of the membranes (called meninges) that surround the brain and spinal cord. Meningitis may be caused by many different viruses and bacteria, or by diseases that can cause inflammation of tissues of the body without infection (such as systemic lupus erythematosus and Behcet's disease). Symptoms of meningitis, which may appear suddenly, often include high fever, severe and persistent headache, stiff neck, nausea, and vomiting. Changes in behavior such as confusion, sleepiness, and difficulty waking up are extremely important symptoms and may require emergency treatment. In infants symptoms of meningitis may include irritability or tiredness, poor feeding and fever. Some types of meningitis can be deadly if not treated promptly.

Primary meningitis may be caused by the following bacteria: Haemophilus influenzae, Neisseria meningitides; Staphylococcus pneumoniae; Streptococcus pyogenes; Escherichia coli; Streptococcus agalactiae; Mycoplasma tuberculosis; Listeria monocytogenes; Cryptococcus neoformans; or by the following viruses: Entervirus; Herpes virus; Adenovirus; Arbovirus; Mumps virus.

Secondary meningitis may be caused by the following bacteria: Candida albicans; Norcardia; Treponema pallidum; Borrelia; Leptospira; Histoplasma capsulatum; Coccidioides immitis; Naegleria fowleri; Acanthamoeba, as well as by HIV or Cytomegalovirus.

### Encephalitis

Encephalitis is an inflammation of the brain. There are many types of encephalitis, most of which are caused by infection by viruses. Symptoms of encephalitis include sudden fever, headache, vomiting, abnormal visual sensitivity to light, stiff neck and back, confusion, drowsiness, clumsiness, unsteady gait, and irritability. Symptoms that require emergency treatment include loss of consciousness, poor responsiveness, seizures, muscle weakness, sudden severe dementia, memory loss, withdrawal from social interaction, and impaired judgement.

### Encephalomyelitis

Encephalomyelitis is the term used to refer to inflammation of both the brain and spinal cord. Encephalomyelitis can be caused by a variety of conditions that lead to irritation of the brain and spinal cord. Among the common causes of encephalomyelitis are viruses which infect the nervous tissues. (An example of a type of virus that can cause encephalomyelitis is the herpes zoster virus.) Persons with encephalomyelitis can exhibit combinations of the various symptoms of either encephalitis or meningitis.

Encephalitis or meningitis are suggested when the symptoms described above are present. Tests that are used in the evaluation of individuals suspected of having encephalitis or meningitis include blood counts, brain scanning (such as MRI scan), and spinal fluid analysis.

The prognosis for encephalitis varies. Some cases are mild, short and relatively benign and patients have full recovery. Other cases are severe, and permanent impairment or death is possible. With early diagnosis and prompt treatment, most patients recover from meningitis. Some forms of bacterial meningitis are very contagious, which makes early and correct diagnosis of the pathogen causing the disease a major public health issue.

It is obvious from this list that the success of treatment will depend on the precise and quick determination of the underlying pathogen.

### Food Poisoning and Diarrhea

Diarrhea generally is defined as frequent and/or loose stools. Diarrhea can be either acute or chronic. Acute diarrhea resolves spontaneously in less than 2-3 weeks while chronic diarrhea lasts longer than 4 weeks. Diarrhea may be caused by such viruses as: Rotavirus, Astrovirus, and Corona virus; or any of the following bacteria: Escherichia coli O0157, Shigella flexneri; Shigella sonnei; Shigella dysenteriae; Shigella boydii; Salmonella typhi; Salmonella enteridis; Salmonella typhimurium; Yersinia enterocolitica; Vibrio cholerae; Listeria monocytogenes; Staphylococcus aureus; Clostridium difficile; Bacillus cereus; Yersinia pseudotuberculosis; Camphylobacter jejunii. However, diarrhea may also be caused by caused by a parasite, Giardia lamblia.

Medications can also be a precipitating cause of diarrhea. Carbohydrates that are poorly absorbed are another source of diarrhea. Many medical conditions can cause chronic diarrhea. Some examples include irritable bowel syndrome, bacterial overgrowth, Crohn's disease, ulcerative colitis, colon cancer, colon polyp (villous adenoma), celiac sprue, hyperthyroidism, Addison's disease, giardiasis and other parasites, medication side effects, laxative abuse, and lactose intolerance. Food poisoning caused by toxins manifests also as diarrhea, that can not be effectively treated with antibiotics.

It is obvious from this list that the success of treatment will depend on the precise and quick determination of the underlying pathogen.

### Sepsis and Sepsis Syndrome

Sepsis is the condition or syndrome caused by the presence of microorganisms or their toxins in the tissue or the bloodstream (septicemia). Sepsis syndrome is a systemic response to infection, defined as hypothermia or hyperthermia, tachycardia, tachypnea, a clinically evident focus of infection or positive blood cultures, one or more end organs with either dysfunction or inadequate perfusion, cerebral dysfunction, hypoxaemia, increased plasma lactate or unexplained metabolic acidosis, and oliguria. It is one of the most common causes of adult respiratory distress syndrome. Sepsis can be caused by such pathogenic bacteria as: Coagulase negative Staphylococci; Staphylococcus aureus; Staphylococcus pneumoniae; Enterococci; Pseudomonas.; Haemophilus influenzae; Bacteroides fragilis; Candida albicans; Salmonella enteritidis; Salmonella typhimurium; S. chloraesuis; Escherichia coli; Citrobacter; Enterobacter; Klebsiella; Serratia marcescens; Streptococcus mitis; Streptococcus sanguis; Streptococcus mutans; Streptococcus salivarius; Streptococcus bovis; Streptococcus adjacens; S. defectives; Streptococcus intermedius; Streptococcus anginosus; Gemella morbillorum; Streptococcus mitior; Streptococcus agalactiae; Haemophilus aphrophilus; Actinobacillus actinomycetem comitans; Campylobacter hominis; Kingella, or Eikevella corrodens. While usually related to infection, septic syndrome can also be associated with noninfectious insults such as trauma, bums, pancreatitis.

It is obvious from this list that the success of treatment will depend on the precise and quick determination of the underlying pathogen.

### Respiratory Infections

Among respiratory infections both acute and chronic the most important are pneumonia, pneumonitis and bronchitis.

Pneumonia is an infection of one or both lungs which is usually caused by a bacteria, virus, or fungus. Currently, over 3 million people develop pneumonia each year in the United States. Over a half a million of these people are admitted to a hospital for treatment. Although most of these people recover, approximately five percent will die from pneumonia. Pneumonia is the sixth leading cause of death in the United States. Most people who develop pneumonia initially have symptoms of a cold which is then followed by a high fever (sometimes as high as 104 degrees), shaking chills, and a cough with sputum production. The sputum is often bloody. Chest pain may develop on one side and the patient may become short of breath. In other cases of pneumonia, there can be a slow onset of symptoms. A worsening cough, headaches, and muscle aches may be the only symptoms. At times, the individual's skin color may change and become dusky or purplish due to their blood being poorly oxygenated. Children and babies often do not have any specific signs of a chest infection, but develop a fever, appear quite ill, and can become lethargic. Elderly people may also have few symptoms with pneumonia.

Among an array of clinical tests sputum samples can be collected and examined under the microscope. If the pneumonia is caused by bacteria, it can often be detected by this examination. A sample of the sputum can be grown in special incubators and the offending bacteria can be subsequently identified. A blood test can be performed (called a CBC) and the white blood cell count of this test can often give a hint as to the severity of the pneumonia and whether it is caused by bacteria or a virus.

Acute respiratory infection such as pneumonia can be caused by such bacteria as: Streptococcus pyogenes; Chlamydia pneumoniae; Neisseria gonorrhoeae; Corynebacterium diphtheriae; Mycoplasma pneumoniae, and Arcanobacterium haemolyticum; or by yeast: Candida; or by such viruses as: Rhinovirus; Corona virus; Herpes simplex virus; Epstein-Barr virus; Adenovirus; Coxsakievirus A; Parainfluenza virus, and Influenza virus. Viral pneumonias do not typically respond to antibiotic treatment. These pneumonias usually resolve over time with the body's immune system fighting off the infection. It is important to make sure that a bacterial pneumonia does not secondarily develop. If it does, then the bacterial pneumonia is treated with appropriate antibiotics. Fungal pneumonias that can occur include actinomycosis, nocardiosis, histoplasmosis, coccidiomycosis, blastomycosis, aspergillosis, and cryptococcosis. These are responsible for a relatively small percentage of pneumonias in the United States. Infections such as bronchitis can also cause coughing, so do certain medications used in treating high blood pressure. Pneumonia can be a serious and life-threatening infection. This is true especially in the elderly, children and those that have other serious medical problems such as emphysema, heart disease, diabetes, and certain cancers, making its correct diagnosis of utmost importance.

Chronic respiratory infection that may manifest itself as chronic cough may me caused by a different group of pathogens, such as: Staphylococcus aureus; Streptococcus pneumoniae; Haemophilus influenzae; Moraxella catarrhalis; Klebsiella pneumoniae; Escherichia coli; Pseudomonas aeruginosa; Legionella; Mycobacterium tuberculosis; Norcardia; Mycoplasma pneumoniae; Chlamydia pneumoniae; Bordetella pertussis; Chlamydia trachomatis; Chlamydia psittaci; Pneumocystis carinii; Respiratory syncytial virus; Parainfluenza virus; Rhinovirus; Adenovirus, or Coxsakievirus A. For correct diagnosis and proper treatment of chronic respiratory infection such as bronchitis doctors have to exclude asthma, postnasal drip, esophageal reflux, drug side effect, or other unusual infections.

It is obvious from this list that the success of treatment will depend on the precise and quick determination of the underlying pathogen.

### Urinary Tract Infections

Urinary tract infections (UTIs) are a serious health problem affecting millions of people each year. Infections of the urinary tract are common—only respiratory infections occur more often. Each year, UTIs account for about 8 million doctor visits.

Not everyone with a UTI has symptoms, but most people get at least some. These may include a frequent urge to urinate and a painful, burning feeling in the area of the bladder or urethra during urination. It is not unusual for a patients to feel bad all over, tired, shaky, washed out, and to feel pain even when not urinating. A fever may mean that the infection has reached the kidneys. Other symptoms of a kidney infection include pain in the back or side below the ribs, nausea, or vomiting. In children, symptoms of a urinary infection may be overlooked or attributed to another disorder. A UTI should be considered when a child or infant seems irritable, is not eating normally, has an unexplained fever that does not go away, has incontinence or loose bowels, or is not thriving.

Urinary tract infections may be caused by the following pathogens: Escherichia coli; Proteus mirabilis; Pseudomonas; Kebsiella; Enterobacter; Enterococci; Staphylococcus aureus; Staphylococcus saprophyticus; Mycobacterium tuberculosis; Candida; Corynebacterium group D2; Ureaplasma urealyticum; Mycoplasma pneumoniae, and Adenovirus.

In the urinalysis test, the urine is examined for white and red blood cells and bacteria. Then the bacteria are grown in a culture and tested against different antibiotics to see which drug the bacteria is sensitive to. Some microbes, like Chlamydia and Mycoplasma, can only be detected with special bacterial cultures. UTIs can be complicated by blockage of the normal urinary flow (obstruction), infection elsewhere or throughout the body (sepsis), and dangerously low blood pressure (shock). Underlying conditions that impair the normal urinary flow (such as kidney stones or diseases of the nervous system leading to bladder weakness) can lead to more complicated UTIs. Curing infections that stem from a urinary obstruction or nervous system disorder depends on finding and correcting the underlying problem, sometimes with surgery. If the root cause goes untreated, this group of patients is at risk of kidney damage. Also, such infections tend to arise from a wider range of bacteria, and sometimes from more than one type of bacteria at a time.

It is obvious from this list that the success of treatment will depend on the precise and quick determination of the underlying pathogen.

### Skin Rashes

Skin rashes, dermatitis can be caused by: Chlamydia psittaci; Mycoplasma pneumoniae; Rickettsia rickettsii; Rickettsia akari; Rickettsia prowazekii; Rickettsia typhi; R. (Orientia) tsutsugamushi; Salmonella typhi; Francisella tularensis; S. mobiliformis; Treponema pallidum; Neisseria gonorrhoeae; Neisseria meningitidis; Leptospira; Listeria monocytosgens; Borrelia burgdorferi; Pseudomonas aeruginosa; Staphylococcus aureus; Streptococcus pyogenes; C. canimorus; Vibrio vulnificus; Candida; Cryptococcus neoformans; Histoplasma capsulatum; Blastomyces dermatitidis; Coccidioides immitis; Plasmodium falciparum; HIV; Echo virus; Coxackievirus; Measles; Adenovirus; Lymphocytic choriomeningitis virus; Dengue virus; Rubella; Yellow fever; Varicella-Zoster; Herpes simplex; Varicella (Chickenpox); Cytomegalovirus; Epstein-Barr virus; Hepatitis B virus; Parvovirus B 19, or Human herpes virus 6.

It is obvious from this list that the success of treatment will depend on the precise and quick determination of the underlying pathogen.

### Sexually Transmitted Diseases

Sexually transmitted diseases (STDs) are infections that can be transferred from one person to another through sexual contact.

STDs can be caused by: Chlamydia trachomatis; Neisseria gonorrhoeae; Mycoplasma; Ureaplasma; Treponema pallidum; Haemophilus ducreyi; Candida; Trichomonas vaginalis; Herpes simplex virus; Herpes virus, and Papilloma virus.

Most STDs are treatable. However, even the once easily cured gonorrhea has become resistant to many of the older traditional antibiotics. Other STDs, such as herpes, AIDS, and genital warts, all of which are caused by viruses, have no cure. Some of these infections are very uncomfortable, while others can be deadly. Syphilis, AIDS, genital warts, herpes, hepatitis, and even gonorrhea have all been known to cause death.

It is obvious from this list that the success of treatment will depend on the precise and quick determination of the underlying pathogen.

### Hepatitis B

Hepatitis B is a virus that causes inflammation of the liver. Hepatitis B virus can cause both an initial (acute) and a chronic form of liver inflammation. The virus is usually transmitted with bodily fluids such as blood, and as such all donor blood has to be tested for its presence. Sexual transmission is believed to be responsible for 30% of the cases worldwide. Complications from hepatitis B are responsible for 1 to 2 million deaths yearly. Liver blood tests become abnormal 1-10 days after infection with the virus. The initial phase of infection lasts a few weeks, and in most people, the infection clears without medications. Chronic hepatitis B is associated with cirrhosis of the liver, liver failure, and liver cancer.

### Hepatitis C

The hepatitis C virus (HCV) is one of the most significant health problems affecting the liver. More than 4 million Americans (1.3% of the U.S. population) and 170 million individuals in the world (3% worldwide) are infected with HCV. One of the major problems with HCV infections is that 85% of individuals initially infected with this virus will become chronically infected, usually for decades. The other 15% of HCV infected individuals simply have an acute infection; that is, one that resolves spontaneously in a few weeks or months. Once established, chronic HCV infection causes an inflammation of the liver called chronic hepatitis. This condition can progress to scarring of the liver (fibrosis), or more advanced scarring (cirrhosis). Some patients with cirrhosis will go on to develop liver failure or the complications of cirrhosis, including liver cancer.

Hepatitis C is transmitted most efficiently through the blood. Therefore, HCV is transmitted by infected blood or blood products, transplantation of infected solid organs (e.g., liver, kidney, heart), and the sharing of contaminated needles among intravenous drug users. All blood donors are currently screened with the following panel: hepatitis C antibodies, hepatitis B surface antigen, hepatitis B core antibodies, elevated alanine aminotransferase, HIV antibodies, and syphilis. As a result, the risk of contracting HCV from a single unit of blood is less than 1:100,000. This risk may be even lower if faster and more sensitive tests that measure hepatitis C viral nucleic acids are universally adopted for blood screening.

### Malaria

Malaria is a parasitic disease that involves infection of the red blood cells. Human malaria is caused by one or more of four species of intraerythrocytic protozoa of the genus Plasmodium (i.e., P. falciparum, P. vivax, P. ovale, or P. malariae). Of the 4 types of malaria, the most serious type is falciparum malaria. It can be life-threatening. The other 3 types of malaria (vivax, malariae, and ovale) are generally less serious and are not life-threatening. The symptoms characteristic of malaria include fever, chills, muscle aches, and headache. Cycles of chills, fever, and sweating that recur every 1, 2, or 3 days are typical. There can sometimes be vomiting, diarrhea, coughing and yellowing jaundice) of the skin and whites of the eyes. Persons with severe falciparum malaria may develop bleeding problems, shock, kidney or liver failure, central nervous system problems, coma, and die. The disease can be transmitted through blood transfusion, and thus necessitates testing of all donor blood.

### Syphilis

Syphilis is an STD that is caused by a spirochete T. pallidum. If left untreated, syphilis can cause extensive damage to the internal organs, such as the brain, and can lead to death. It can be transmitted through blood transfusion. All blood donors are currently screened with the following panel: hepatitis C antibodies, hepatitis B surface antigen, hepatitis B core antibodies, elevated alanine aminotransferase, HIV antibodies, and syphilis.

### HIV

Acquired immune deficiency syndrome (AIDS) is a contagious disease caused by the human immunodeficiency virus (HIV). HIV infection weakens the body's immune system and increases the body's vulnerability to many different infections as well as to the development of certain cancers. AIDS is one of the most frightening of the STDs because it is the most uniformly fatal of the group.

Currently, it is believed that AIDS can only be transmitted through bodily secretions, such as blood, semen, or vaginal secretions. All blood donors are currently screened for HIV antibodies.

### HTLV

Human T-cell leukemia virus (HTLV) is one of a group of retroviruses which causes the disease T-cell leukemia in humans. T-cell leukemia is a type of the cancer where the body uncontrollably produces large amounts of abnormal T lymphocytes. All blood donors have to be screened for HTLV.

### Ear Infections

Among ear infections otitis media is the most prominent. Otitis media is inflammation of the middle ear. Otitis media can be acute or chronic. Acute otitis media is usually of rapid onset and short duration. Acute otitis media typically causes fluid accumulation in the middle ear together with signs or symptoms of ear infection; a bulging eardrum usually accompanied by pain, or a perforated eardrum, often with drainage of purulent material (pus). Chronic otitis media is a persistent inflammation of the middle ear. This condition can cause ongoing damage to the middle ear and eardrum and there may be continuing drainage through a hole in the eardrum. Otitis media is the most common diagnosis in sick children in the U.S. Young children, infants, and preschoolers are particularly prone. Almost every child has at least one bout of acute otitis media before the age of 6. Serous otitis media is inflammation in the middle ear without infection. If left untreated ear infection can lead to a diminished hearing or even deafness.

Ear infections can be caused by: Streptococcus pneumoniae; Haemophilus influenzae; Moraxella catarrhalis; Pseudomonas aeruginosa; Aspergillus; Candida; Respiratory syncytial virus; Influenza virus; Enterovirus; Rhinovirus.

It is obvious from this list that the success of treatment will depend on the precise and quick determination of the underlying pathogen.

### Eye Infections, Conjunctivitis

Conjunctivitis or pink eye refers to a redness or irritation of the membranes on the inner part of the eyelids and the membranes (conjunctiva) covering the whites of the eyes. These membranes react to a wide range of bacteria, viruses, allergy-provoking agents, irritants, and toxic agents, as well as to underlying diseases within the body. Viral and bacterial forms of conjunctivitis are common in childhood. Overall however, there are many causes of pink eye. These can be classified as either infectious or noninfectious.

Eye infections may be caused by: Streptococcus pneumoniae; Streptococcus aureus; Staphylococcus; Haemophilus influenzae; Pseudomonas; Moraxella catarrhalis; Chlamydia trachomatis; Neisseria gonorrhoeae; Acanthamoeba; Adenovirus, and Cytomegalovirus. Viral and bacterial pink eye is highly contagious.

Allergic pink eye, chemical pink eye can present similar symptoms as bacterial and viral, but are not contagious. Persistent conjunctivitis can be a sign of an uncommon underlying illness in the body. Most often these are rheumatic diseases, such as rheumatoid arthritis and systemic lupus erythematosus. Conjunctivitis is also seen in Kawasaki's disease (a rare disease associated with fever in infants and young children) and certain inflammatory bowel diseases such as ulcerative colitis and Crohn disease.

It is obvious from this list that the success of treatment will depend on the precise and quick determination of the underlying pathogen.

A variety of pathogenic microorganisms can also cause fever of unknown origin, arthritis, heartache, etc. The clinical panel of the present invention encompasses but is not limited to the above mentioned diseases, and also includes any disease that may be caused by a pathogenic microorganism.

### Mitochondrial Diseases

Human mtDNA consists of 16,569 base pairs of double-stranded, circular DNA. Encoded on the mt genome are 13 polypeptides involved in the electron transport chain, 2 ribosomal RNAs (rRNAs), and 22 transfer RNAs (tRNAs). Due in part to poor repair mechanisms, mtDNA accumulates mutations more rapidly than does nuclear DNA and thus is highly polymorphic (Simon and Johns 1999 Annu Rev Med 50:111-127). The first pathogenic mtDNA mutations were identified in 1988. A missense mutation at nucleotide position (np) 11778 was found in association with Leber's hereditary optic neuropathy (LHON) (Wallace, D. C. et al. 1988 Science 242:1427-30), and deletions of mtDNA were associated with mt myopathies (Holt, I. J. et al. 1988 Nature 331:717-19) and Kearns-Sayre syndrome (KSS) (Lestienne, P. & Ponsot, G. 1988 Lancet 1:885; Zeviani, M. et al. 1988 Neurology 38:1339-46). Soon thereafter, duplications of mtDNA were identified in two patients with mt myopathies (Poulton, J. et al. 1989 Lancet 1:236-40). Since then, there has been a rapid rise in the number of mtDNA mutations identified in association with clinical disorders, although in some cases, the pathogenicity of the putative mutations is not well established. MtDNA mutations have been identified in each type of mt gene (protein-coding, tRNA, and rRNA). Epidemiological studies have revealed that mtDNA disorders are far more common than previously believed. In patients with diabetes mellitus, hearing loss, or renal failure, the prevalence of MELAS (mitochondrial encephalopathy, lactic acidosis, and strokelike syndrome) has been estimated to be as high as 1% to 10%. Manifestations of mtDNA disorders such as diabetes mellitus present identically to idiopathic disease. Thus, internists will likely encounter patients with unrecognized mtDNA disorders, necessitating genetic testing. It has been suggested that early treatment of mtDNA disorders might prevent the development of irreversible disease, such as stroke, in affected patients.

Over the past decade, mtDNA mutations have been associated with diverse clinical phenotypes (Table 1). Neurologic manifestations often are prominent, including seizures, strokes (especially in a young person), dementia, ataxia, optic neuropathy, retinopathy, sensorineural hearing loss, peripheral neuropathy, and myopathy. Common cardiac features include a cardiomyopathy and conduction defects. Endocrine abnormalities, especially diabetes mellitus, are frequent. Gastrointestinal manifestations include colonic pseudo-obstruction, hepatopathy, and weight loss. The most prominent renal manifestation is a glomerulopathy that resembles Fanconi's syndrome. Bone marrow dysfunction can occur with a prominent sideroblastic anemia.

TABLE 1
Clinical and laboratory features of some mitochondrial disorders

| | | LHON | MELAS | MERRF | CPEO | Pearson's | NARP | MILS |
|---|---|---|---|---|---|---|---|---|
| CNS | developmental | − | + | +/− | + | − | − | + |
| | delay/regression | | | | | | | |
| | seizures | − | + | + | − | − | − | +/− |
| | ataxia | − | + | + | + | − | + | +/− |
| | myoclonus | − | +/− | + | − | − | − | − |
| | stroke-like | − | + | − | − | − | − | − |
| | episodes | | | | | | | |
| | dystonia | +/− | +/− | − | − | − | − | + |
| PNS | peripheral | − | +/− | +/− | +/− | − | + | − |
| | neuropathy | | | | | | | |
| Muscle | myopathy | − | − | + | − | − | − | − |
| Eye | ophthalmoplegia | − | − | − | + | +/− | − | − |
| | pigmentary | − | − | − | + | − | + | +/− |
| | retinopathy | | | | | | | |
| | optic atrophy | + | − | − | − | − | +/− | +/− |
| | ptosis | − | + | − | + | − | − | − |
| Heart | cardiomyopathy | − | +/− | − | +/− | − | − | +/− |
| | conduction | − | +/− | − | + | − | − | − |
| | abnormality | | | | | | | |
| Inner ear | SNHL | − | + | + | + | − | +/− | − |
| Pancreas | DM | − | +/− | − | +/− | − | − | − |
| | exocrine | − | − | − | − | + | − | +/− |
| | dysfunction | | | | | | | |
| Marrow | sideroblastic | − | − | − | +/− | + | − | − |
| | anemia | | | | | | | |
| Kidney | fanconi | − | +/− | − | +/− | +/− | − | − |
| | syndrome | | | | | | | |
| Labs | lactic acidosis | + | + | + | + | + | − | +/− |
| | serum or CSF | | | | | | | |
| | RRFs (muscle | − | + | + | + | +/− | − | − |
| | bx) | | | | | | | |
| Genetics | missense | + | − | − | − | − | + | + |
| | mutation | | | | | | | |
| | mt tRNA | − | + | + | − | − | − | − |
| | mutation | | | | | | | |
| | mt DNA deletion | − | − | − | + | + | − | − |
| | nuclear DNA | − | − | − | +/− | +/− | − | − |
| | mutation | | | | | | | |

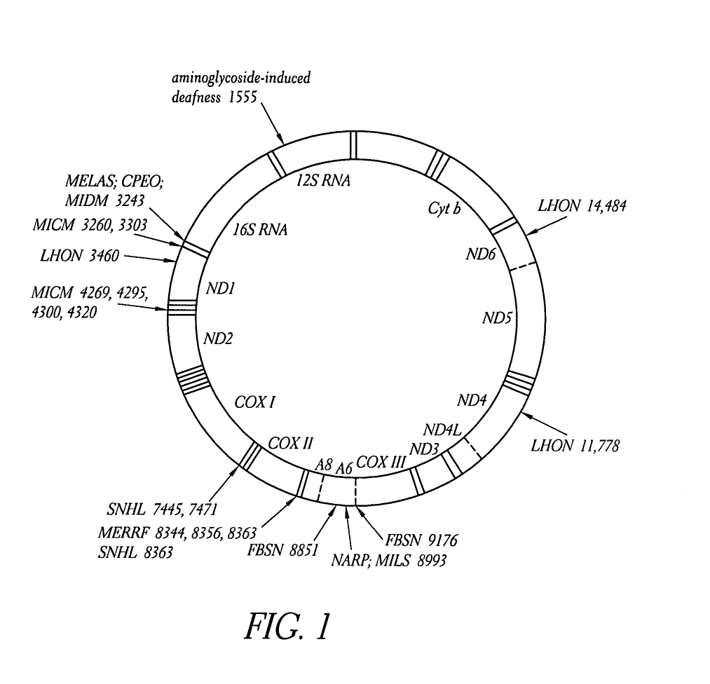

Several distinct combinations of these characteristics have been identified in association with specific mtDNA mutations (FIG. 1). However, a particular mutation may be associated with different phenotypes in different patients (clinical heterogeneity), and a single clinical phenotype may be associated with different mtDNA mutations in different patients (genetic heterogeneity). Nonetheless, the presence of a recognizable clinical syndrome often helps focus the search for likely mtDNA mutations.

### Leber's Hereditary Optic Neuropathy

Leber's Hereditary Optic Neuropathy (LHON) is clinically distinct from other mt disorders, the main phenotype being subacute, painless, bilateral visual loss characterized by central scotomas, abnormal color vision, and optic atrophy (Johns, D. R. 1995 N Engl J Med 333:638-44). The age of onset of vision loss averages 23 years but ranges from 8 to 60 years (Wallace, D. C. 1992 Science 256:628-32; Johns, D. R. 1994 Clin Neurosci 2:146-50). Affected males are 3-4 times as common as affected females (Johns, D. R. 1994 Clin Neurosci 2:146-50). The visual loss in LHON provides an example of the interactions between mtDNA mutations and other mt genetic factors, nuclear genes, and environmental factors. Three mutations, at np 3460, 11778, and 14484, are primary pathogenetic mutations and together account for the majority of LHON cases (Savontaus, M. L. 1995 Biochim Biophys Acta 1271:261-63). Several other mtDNA mutations, including ones at np 4216 and 13708 (see Table 2), appear to play secondary roles in LHON by increasing the likelihood of expression of a primary LHON mutation when both are present (Johns, D. R., Berman, J. 1991 Biochem Biophys Res Commun 174:1324-30; Brown, M. D. et al. 1997 Am J Hum Genet 60:381-87).

### Mt Encephalomyopathy, Lactic-Acidosis, and Stroke-Like Episodes

Mt Encephalomyopathy, Lactic-Acidosis, and Stroke-Like Episodes (MELAS) is the most common of the genetically defined, maternally inherited mt disorders. The syndrome is characterized by encephalopathy and subacute stroke-like events. Other common clinical features include migraine-like headaches, recurrent vomiting, weakness of extremities, and short stature (Pavlakis, S. G. et al. 1984 Ann Neurol 16:481-88). A missense mutation in the tRNALeu (UUR) gene is present in 80% of MELAS patients. This mutation demonstrates clinical heterogeneity, as it is associated with multiple other phenotypes, including chronic progressive external ophthalmoplegia (CPEO), myopathy, deafness, maternally inherited diabetes mellitus, and dystonia. However, there are other mtDNA mutations that are associated with this syndrome (see Table 2). Occasionally MELAS syndrome and Herpes simplex encephalitis (HSE) may have a similar clinical presentation necessitating differential diagnosis (de Toledo, M. et al. 2001 Rev Neurol 33:148-50).

### Myoclonic Epilepsy with Ragged-Red Fibers

Clinical features of myoclonic epilepsy with ragged-red fibers (MERRF) include myoclonus, seizures, ataxia, and mt myopathy, in addition to features common to other mt disorders (Silvestri, G. et al. 1993 Neurology 43:1200-6). Maternal relatives may be asymptomatic or have partial clinical syndromes, including multiple lipomas and cardiovascular disease. Most patients with MERRF harbor a heteroplasmic mutation at np 8344 in the tRNALys gene (Silvestri, G. et al. 1993 Neurology 43:1200-6). Two other mutations in the same gene, at np 8356 (25) and 8363 (26), also have been linked to MERRF. Four other mutations in tRNA genes have been associated with MERRF/MELAS overlap syndromes (Zeviani, M. et al. 1993 Eur J Hum Genet 1:80-87; Moraes, C. T. et al. 1993, J Clin Invest 92:2906-15; Folgero, T. et al. 1995 Eur Neurol 35:168-71; Nakamura, M. et al. 1995 Biochem Biophys Res Commun 214:86-93).

### Chronic Progressive External Ophthalmoplegia

Chronic Progressive External Ophthalmoplegia (CPEO) is characterized by ptosis, weakness of extraocular muscles, and limb weakness. Other features of mt disorders also may be present, including features of MELAS or MERRF. Kearns-Sayre syndrome (KSS), a form of CPEO that begins prior to age 20, is characterized by an atypical pigmentary retinopathy, ataxia, elevated cerebrospinal fluid protein, and heart block (DiMauro, S. & Moraes, C. T. 1993 Arch Neurol 50:1197-208). CPEO is linked to either large deletions in mtDNA (Holt, I. J. et al. 1988 Nature 331:717-19) or to various point mutations in tRNA molecules, including the np 3243 mutation more commonly associated with MELAS (Moraes, C. T. et al. 1993 Neuromuscul Disord 3:43-50). The single deletions arise sporadically, whereas multiple mtDNA deletions can be inherited as an autosomal dominant trait (Zeviani, M. et al. 1989 Nature 339:309-11; Carrozzo, R. et al. 1998 Neurology 50:99-106). About a third of CPEO/KSS patients carry the same deletion, a 4977-base-pair deletion known as the common deletion (Schon, E. A. et al. 1989 Science 244:346-49). In addition, rare patients have been identified with partially duplicated mtDNA molecules (Poulton J. 1992 J Inherit Metab Dis 15:487-98). Thus, CPEO provides a good example of genetic heterogeneity, with several distinct types of mutations giving rise to similar clinical phenotypes.

### Neuropathy, Ataxia, and Retinitis Pigmentosa; Maternally Inherited Leigh Syndrome; and Familial Bilateral Striatal Necrosis

Neuropathy, ataxia, and retinitis pigmentosa (NARP) is characterized by seizures, dementia, ataxia, retinal pigmentary changes, sensory neuropathy, and neurogenic proximal muscle weakness in association with missense mutations at np 8993 in the ATPase 6 gene (Holt, I. J. et al. 1990 Am J Hum Genet 46:428-33). In NARP, these heteroplasmic mutations are present at a modest mutational burden of about 70%. However, at a higher mutational burden (greater than 90%), the same mutation is associated with the clinically distinct syndrome of maternally inherited Leigh syndrome (MILS) (Santorelli, F. M. et al. 1993 Ann Neurol 34:827-34).

MILS is a severe, multisystem, degenerative disorder with onset usually in the first year of life. Affected infants manifest hypotonia, psychomotor regression, seizures, myoclonus, ataxia, brainstem dysfunction, optic atrophy, and peripheral neuropathy.

Familial bilateral striatal necrosis (FBSN) has clinical features similar to those of MILS but is less severe. FBSN has been linked to point mutations at np 8851 (De Meirleir, L. et al. 1995 Pediatr Neurol 13:242-46) and 9176 (Thyagarajan, D. et al. 1995 Ann Neurol 38:468-72) in the ATPase 6 gene, as well as other mutations (see Table 2). Both MILS and FBSN are associated with symmetric foci of necrosis in the basal ganglia, but in FBSN, the thalamus and brainstem are relatively spared.

### Sensorineural Hearing Loss

Sensorineural hearing loss (SNHL) is a common feature of many mt disorders, including MERRF and MELAS. In addition, some mtDNA mutations are associated with maternally inherited SNHL as the major or exclusive symptom. Three such mutations are found in tRNA genes at np 7445 (Reid, F. M. et al. 1994 Hum Mutat 3:243-47), 7471 (Tiranti, V. et al. 1995 Hum Molec Genet 4:1421-27), and 8363 (Santorelli, F. M. et al. 1996 Am J Hum Genet 58:933-39). A fourth mutation linked to SNHL is located at np 1555 in the 12S rRNA gene. Patients carrying this mutation are highly susceptible to aminoglycoside-induced deafness, although in some pedigrees, deafness can occur without known antibiotic exposure (Prezant, T. R. et al. 1993 Nat Genet 4:289-94; Hutchin, T. et al. 1993 Nucl Acids Res 21:4174-79; Inoue, K. et al. 1996 Biochem Biophys Res Commun 223:496-501).

### Pearson's Marrow/Pancreas Syndrome

In 1979, Pearson et al. (Pearson, H. A. et al. 1979 J Pediatr 95:976-84) described a series of children with sideroblastic anemia and exocrine pancreatic dysfunction. Like CPEO/KSS, Pearson's syndrome is linked to large-scale deletions of mtDNA (Rotig, A. et al. 1989 Lancet 1:902-3). The few patients with Pearson's syndrome who survive eventually develop KSS (McShane, M. A. et al. 1991 Am J Hum Genet 48:39-42).

### Maternally Inherited Diabetes Mellitus

Endocrinopathies are common features of many mt diseases. Diabetes mellitus can be a component of the MELAS syndrome associated with the np 3243 mutation (Onishi, H. et al. 1993 J Neurol Sci 114:205-8). However, maternally inherited adult-onset type II non-insulin-dependent diabetes mellitus (NIDDM) also can be the exclusive feature of this mutation, usually when it is present at a lower mutational burden (Reardon, W. et al. 1992 Lancet 340:1376-79; Gerbitz, K. D. et al. 1993 FEBS Lett 321:194-96). The 3243 mutation is estimated to account for as much as 1-2% of all NIDDM patients (Kadowaki, H. et al. 1993 Lancet 341:893-94).

### Maternally Inherited Cardiomyopathy

Like diabetes, cardiomyopathy may be a feature of other mt syndromes, including MERRF and MELAS (Anan, R. et al. 1995 Circulation 91:955-61), but several mtDNA mutations are associated with cardiomyopathy in the absence of other clinical abnormalities (Schon, E. A. et al. 1997 J Bioenerg Biomemb 29:131-49). These mutations are located in tRNA genes, including four within the same gene (tRNAIle).

### MtDNA Mutations Secondary to Mutations in Nuclear DNA

Several disorders associated with multiple mtDNA deletions can be inherited in an autosomal dominant or autosomal recessive manner, indicating that a nuclear DNA mutation predisposes to mtDNA deletions. These disorders include CPEO (see above); mt neurogastrointestinal encephalomyopathy (MNGIE); and diabetes insipidus, diabetes mellitus, optic atrophy, and deafness (DIDMOAD or Wolfram syndrome).

MNGIE syndrome consists of leukoencephalopathy, external ophthalmoplegia, peripheral neuropathy, and intestinal dysmotility (Bardosi, A. et al. 1987 Acta Neuropathol 74:248-58). MNGIE is inherited as an autosomal recessive trait. In one study, 4 of 8 patients had multiple deletions in mtDNA (Hirano, M. et al. 1994 Neurology 44:721-27). The key features of DIDMOAD are diabetes insipidus, diabetes mellitus, optic atrophy, and sensorineural deafness. This disorder usually is inherited in an autosomal recessive manner and has been associated with heteroplasmic, large deletions of mtDNA, although it also has been reported in association with point mutations in mtDNA (Barrett, T. G. & Bundey, S. E. 1997 J Med Genet 34:838-41). Thus, CPEO, MNGIE, and DIDMOAD can be associated with nuclear DNA mutations that predispose to deletions of mtDNA.

### Other Disorders of mtDNA

Since the discovery of the first pathogenic mtDNA mutations in 1988, the rate of discovery of novel mtDNA mutations and new clinical phenotypes has continued to increase. MtDNA mutations have been linked to dystonia (Jun, A. S. et al. 1994 PNAS USA 91:6206-10; De Vries, D. D. et al. 1996 Am J Hum Genet 58:703-11), chorea (Nelson, I. et al. 1995 Ann Neurol 37:400-3), and various other clinical phenotypes.

Evidence is accumulating that, in addition to its role in relatively rare, classic mt disorders, oxidative stress due to mt dysfunction plays a role in the pathogenesis of more common late-onset neurodegenerative disorders, including Parkinson's disease, (PD), Alzheimer's disease (AD), Huntington's disease (HD), amyotrophic lateral sclerosis (ALS), and even the process of aging itself (Wallace, D. C. 1992 Science 256:628-32; Beal, M. F. 1995 Ann Neurol 38:357-66). Oxidative stress is the increased generation of free radicals resulting in oxidative damage to DNA, proteins, and lipids. Impairment of mt oxidative phosphorylation is associated with increased oxidative stress.

Evidence of defects in mt energy metabolism is particularly compelling in the case of PD. Activity of complex I of the mt electron transport chain is decreased in platelets, muscle, and substantia nigra of PD patients (Schulz, J. B. & Beal, M. F. 1994 Curr Opin Neurol 7:333). Recent data suggest that the complex I defects in PD patients result from defects in mtDNA. Although several studies have attempted to identify mtDNA mutations in PD patients (Bandmann, O. et al. 1998 Mov Disord 13:203-11), the specific mtDNA mutations identified thus far are uncommon, and none has been shown to account for the complex I defect or to be pathogenic. Evidence of defects in energy metabolism also exists for AD. Activity of complex IV of the electron transport chain is decreased and markers of oxidative stress are increased in the cerebral cortex of AD patients (Beal, M. F. 1995 Ann Neurol 38:357-66). The increase in oxidative damage to mtDNA is much higher than the increase in nuclear DNA in AD brain tissue (Meccoci, P. et al. 1994 Ann Neurol 36:747-51). A recent report suggested that several heteroplasmic point mutations in mtDNA-encoded subunits of complex IV were present at increased mutational burdens in AD patients (Davis, R. E. et al. 1997 PNAS USA 94:4526-31). Thus, there is evidence of mtDNA abnormalities in both PD and AD, but specific mtDNA mutations to account for mt dysfunction in these diseases have yet to be definitively identified.

Perhaps the most common manifestation of mt dysfunction is the process of aging. Age-related mt dysfunction due to cumulative oxidative damage to mtDNA and other macromolecules is hypothesized to be a major contributor to cellular aging and may be important in many age-related disorders (Shigenega, M. K. et al. 1994 PNAS USA 91:10771-78). Oxidative damage accumulates much more rapidly in mtDNA than in nuclear DNA (Meccoci, P. et al. 1993 Ann Neurol 34:609-16). Furthermore, a 4977-base-pair mtDNA deletion (the “common deletion”) that is present at low mutational burdens in human mtDNA increases 1000- to 10,000-fold during a normal human lifespan, although it normally reaches only about 0.1% of mtDNA in aged muscle (Cortopassi, G. et al. 1992 PNAS USA 89:7370-74; Siamonetta, S. et al. 1992 Biochim Biophys Acta 1180:113-22).

The pattern of clinical features, if it indicates a known mt syndrome, can direct the search for specific mtDNA mutations. Genetic tests are commercially available for the most common mutations associated with LHON, MELAS, MERRF, and some of the other mt syndromes. Several research laboratories can assist with evaluation for more rare mtDNA mutations. A differential diagnosis of a mt genetic disorder is important in genetic counseling as well as in choosing among treatment options. It is important to diagnose mitochondrial disorders to provide genetic counseling to affected families and because of the possible benefits of early therapy.

### Primary Blood Groups

A system of describing the oligosaccharide antigens found on the surface of human blood cells. According to the type of antigen present, a person may be assigned a blood type of A, B, AB cis AB or O. A second type of antigen, the Rh factor, renders a “positive” or “negative” blood type. The ABO blood group system is important because it determines who can donate blood to or accept blood from whom. Type A or AB blood will cause an immune reaction in people with type B blood, and type B and AB blood will cause a reaction in people with type A blood. Conversely, type O blood has no A or B antigens, so people with type O blood are “universal donors.” Since AB blood already produces both antigens, people who are type AB can accept any of the other blood types without suffering an immune reaction. Cis AB blood group is a rare variant of the AB blood group resulting from inheritance of both A and B genes on one chromosome. It may lead to misclassification in ABO grouping and clinical misdiagnosis as a result of its divergence from the laws of Landsteiner and Mendel. Rh (“rhesus”) factor is an antigen that may or may not be present on the surface of human blood cells. If a person's blood has the antigen, their blood type is “positive”; if they do not, it is “negative.” The Rh factor is important mainly because if a woman who is Rh⁻ conceives a child who is Rh⁺, the mixing of their bloods in the placenta may provoke an immune reaction in the mother that can cause a life-threatening agglutination of the fetus' blood cells. Rhesus factor subtypes include: Rh D, Rh E, Rh e, Rh C, and Rh c subtypes.

### C. DNA Chips

Any suitable microarray chips can be used in the present clinical panel. In a specific embodiment, microarray chips suitable for nucleic acid analysis, e.g., gene chip, and/or DNA chip, can be used in the present device (See generally, Ausubel et al. 2000 Current Protocols in Molecular Biology, §22, John Wiley & Sons, Inc.; and Schena (ed.) 2000 Microarray Biochip Technology, Eaton Publishing Company/Bio Techniques Books Division). In a specific embodiment, the microarray chips disclosed in the following U.S. Pat. Nos. can be used in the present device: 6,245,511; 6,242,246; 5,827,651; U.S. Patent publication US 2001/0016321; PCT publications WO 01/68915; WO 02/02794; WO 00/73504; and WO 01/81621.

The microarray chip can have any desirable densities. In a specific embodiment, the microarray chips have a density of (10)ₙ spots per chip surface, wherein n is an integer that is at least 1. In another embodiment, n is an integer that is at least 2, at least 4, at least 8, at least 10, at least 20, at least 40.

In another specific embodiment, the microarray chip has attached thereto a plurality of target moieties. The microarray chip of the present invention can have attached thereto a plurality of target moieties in facing up or down direction. The target moieties can be attached to the microarray chip using any suitable methods. The target moieties can be attached to the microarray chip covalently, non-covalently, through specific or non-specific linkage, can be attached directly or through a linker. The linker can be sensitive to certain treatment, such as physical, chemical or enzymatic treatment.

Any suitable target moieties can be attached to the microarray chip. The target moieties can be pure substances or composite materials, can be chemical or biological materials, or can be synthetic or isolated/purified from biological sources or samples. Exemplary target moieties include cells, cellular organelles, viruses, molecules and an aggregate or complex thereof. Non-limiting examples of attachable molecules include amino acids, peptides, proteins, nucleosides, nucleotides, oligonucleotides, nucleic acids, vitamins, monosaccharides, oligosaccharides, carbohydrates, lipids or a complex thereof.

Any nucleic acids, including single-, double-, and triple-stranded nucleic acids, can be attached to the microarray chip. Examples of such nucleic acids include DNA, such as A-, B- or Z-form DNA, and RNA such as mRNA, tRNA and rRNA.

Any nucleosides can be attached to the microarray chip. Examples of such nucleosides include adenosine, guanosine, cytidine, thymidine and uridine. Any nucleotides can be attached to the microarray chip. Examples of such nucleotides include AMP, GMP, CMP, UMP, ADP, GDP, CDP, UDP, ATP, GTP, CTP, UTP, dAMP, dGMP, dCMP, dTMP, dADP, dGDP, dCDP, dTDP, dATP, dGTP, dCTP and dTTP.

In a specific embodiment said attached target moieties are oligonucleotides. In a specific embodiment the oligonucleotides are probes complementary to test moieties. In a specific embodiment test moieties are nucleic acids of human pathogens. In another embodiment test moieties are human genes. In another embodiment test moieties are human mitochondrial DNA. Non-limiting examples of human pathogens are: fungi, bacteria, and viruses. Fungus, bacterium cells, or viruses can belong to any genus or subgenus of the fungus, bacterium, or viral kingdom. Ciliates, flagellates and microsporidia are also included in the list of human pathogens.

When DNA-DNA, DNA-RNA, RNA-RNA interactions are to be detected, the contacting, i.e., hybridizing step, can be conducted under suitable condition, e.g., under low, middle or high stringency.

The interaction between said test moiety and said plurality of target moieties can be detected by any suitable methods. For example, the test moiety and/or target moieties can be labeled to facilitate detection. Any suitable label can be used. Exemplary labels include a radioactive, a fluorescent, a chemical, an enzymatic, a luminescent and a FRET (fluorescence resonance energy transfer) label. The luminescent label can be a chemiluminescent label or a bioluminescent label. The labels can be attached or conjugated, directly or indirectly, to the test moiety alone, the target moiety alone, or on both. The read-out can be a positive or a negative signal. Any suitable assay formats, including sandwich or competitive formats, can be used.

The process for preparing DNA chip contained in said clinical panel comprises the steps of: a) preparing DNA probes which have nucleotide sequences complementary to DNA or RNA of pathogens or human genes, or human mitochondrial DNA, and b) affixing the DNA probes to a solid surface.

The production of biochips on which nucleic acids are immobilized is well known in the art. The biochip may be a Langmuir-Bodgett film, functionalized glass, germanium, silicon, PITE, polystyrene, gallium arsenide, gold, silver, or any other material known in the art that is capable of having functional groups such as amino, carboxyl, thiol or hydroxyl incorporated on its surface. Preferably, these groups are then covalently attached to crosslinking agents, so that the subsequent attachment of the nucleic acid ligands and their interaction with target molecules will occur in solution without hindrance from the biochip. Typical crosslinking groups include ethylene glycol oligomer, diamines, and amino acids. Any suitable technique useful for immobilizing a nucleic acid ligand to a biochip is contemplated by this invention. In one embodiment, one or more nucleic acid ligands will be attached to the support by photolithography using a photoreactive protecting group on a coupling agent. Such a technique is disclosed in McGall et al. U.S. Pat. No. 5,412,087. In another embodiment, the nucleic acid ligand array will be formed on the biochip by an “ink-jet” method, whereby the ligands are deposited by electro-mechanical dispensers at defined locations. An ink-jet dispenser capable of forming arrays of probes with a density approaching one thousand per square centimeter is described in Hayes et al. U.S. Pat. No. 5,658,802.

The method for diagnosis of disease using clinical panel of the invention comprises the steps of: a) amplifying DNA obtained from clinical samples by PCR with primers of the clinical panel; b) applying the amplified DNA to the DNA chip of the clinical panel to hybridize the amplified DNA with DNA probes of the DNA chip; and, c) detecting DNA bound on the surface of the DNA chip after labeling hybridized DNA.

Test DNA can be purified from clinical samples by any means known in the art including, but not limited to those described, e.g., in Sambrook et al. 1989 Molecular Cloning: A Laboratory Manual, 2nd edition, Cold Spring Harbor Press, Cold Spring Harbor Laboratory, N.Y.; Ausubel et al. (eds.), 1987 Current Protocols in Molecular Biology, Greene Publishing Associates, Brooklyn, N.Y.; as well as in, e.g., U.S. Pat. No.: 4,935,342). Numerous commercial kits are available for DNA purification including QIAGEN Maxi- and MiniPrep.

Clinical samples include, but not limited to: cerebro-spinal fluid; blood, sputum, hemoptysis, stool, urine, skin, urethral discharge, vaginal swab, ear discharge, eye discharge, tears, exudates, organ tissue samples, and the like.

Amplification of DNA from target samples can be accomplished by e.g., PCR (see generally H. A. Erlich (ed.) 1992 PCR Technology: Principles and Applications for DNA Amplification Freeman Press, N.Y.; Innis, et al. 1990 PCR Protocols: A Guide to Methods and Applications Academic Press San Diego Calif.; Mattila et al. 1991 Nucleic Acids Res 19:4967; Eckert et al. 1991 PCR Methods and Applications 1:17; McPherson et al. (eds.) PCR IRL Press, Oxford; and U.S. Pat. No. 4,683,202. Other suitable amplification methods include the ligase chain reaction (LCR) (see Wu and Wallace 1989 Genomics 4:560; Landegren et al 1988 Science 241:1077); transcription amplification (Kwoh et al. 1989 PNAS USA 86:1173); and self-sustained sequence replication (Guatelli et al. 1990 PNAS USA 87:1874), and nucleic acid based sequence amplification (NASBA). The latter two amplification methods involve isothermal reactions based on isothermal transcription, which produce both single-stranded RNA (ssRNA) and double-stranded DNA (dsDNA) as the amplification products in a ratio of about 30 or 100 to 1, respectively.

Nucleic acid probes and primers based on sequences of known pathogens and human DNA and human mtDNA can be prepared by standard techniques. Such a probe or primer comprises an isolated nucleic acid. In the case of probes, the nucleic acid may further comprise a label (e.g., a radionuclide such as ³²P-ATP or ³⁵S) or a reporter molecule (e.g., a ligand such as biotin or an enzyme such as horseradish peroxidase). The [³²P]-ATP, [³⁵S]-dATP and [³⁵S]-methionine can be purchased, for example, from DuPont NEN (Wilmington, Del.). Probes are used to identify the presence of a hybridizing nucleic acid sequence, e.g., a DNA in a sample. Primers are used, for amplification of nucleic acid sequences, e.g., by the polymerase chain reaction (PCR). See, e.g., Innis et al. (eds.) 1990 PCR Protocols: A Guide to Methods and Applications, Academic Press: San Diego. The preparation and use of probes and primers is described, e.g., in Sambrook et al. (1989) or Ausubel et al. (1987).

Useful primers and probes can be 8 to 12, or 13 to 15, or 16, 17, 18, 19, or 20 bases in length. Other useful primers and probes can be longer or shorter than these, depending on their intended use. Accordingly, embodiments of the invention include primers and probes of about 25, 30, 40, 60, 80, 100, or 150 bases, or longer.

In another aspect, the invention provides a method for diagnosing of human disease by detecting interaction between a test moiety and a plurality of target moieties, which method comprises: a) providing a clinical panel of the present invention which comprises a microarray chip, wherein said microarray chip has a plurality of target moieties attached to it; b) contacting a test moiety with said plurality of target moieties provided in step a); and c) detecting interaction between said test moiety and said plurality of target moieties. The present method can be used in diagnosis, genetic screening, etc.

Any suitable clinical panel, including the panels described below, can be used in the present method.

In a specific embodiment, the clinical panel comprises a microarray chip with a plurality of oligonucleotides complimentary to nucleic acids of pathogens that cause symptoms associated with meningitis.

In another embodiment, the clinical panel comprises a microarray chip with a plurality of oligonucleotides complimentary to nucleic acids of pathogens that cause symptoms associated with diarrhea.

In another embodiment, the clinical panel comprises a microarray chip with a plurality of oligonucleotides complimentary to nucleic acids of pathogens that cause symptoms associated with sepsis.

In another embodiment, the clinical panel comprises a microarray chip with a plurality of oligonucleotides complimentary to nucleic acids of pathogens that cause symptoms associated with acute respiratory infection.

In another embodiment, the clinical panel comprises a microarray chip with a plurality of oligonucleotides complimentary to nucleic acids of pathogens that cause symptoms associated with chronic respiratory infection.

In another embodiment, the clinical panel comprises a microarray chip with a plurality of oligonucleotides complimentary to nucleic acids of pathogens that cause symptoms associated with urinary tract infection.

In another embodiment, the clinical panel comprises a microarray chip with a plurality of oligonucleotides complimentary to nucleic acids of pathogens that cause symptoms associated with rash and dermatitis.

In another embodiment, the clinical panel comprises a microarray chip with a plurality of oligonucleotides complimentary to nucleic acids of pathogens that cause symptoms associated with STDs.

In another embodiment, the clinical panel comprises a microarray chip with a plurality of oligonucleotides complimentary to nucleic acids of pathogens that cause symptoms associated with ear infections.

In another embodiment, the clinical panel comprises a microarray chip with a plurality of oligonucleotides complimentary to nucleic acids of pathogens that cause symptoms associated with eye infections.

In another embodiment, the clinical panel comprises a microarray chip with a plurality of oligonucleotides complimentary to nucleic acids of pathogens that cause symptoms associated with fever of unknown origin.

In another embodiment, the clinical panel comprises a microarray chip with a plurality of oligonucleotides complimentary to human DNA encoding blood groups as well as to nucleic acids of pathogens that can be transmitted through blood.

In another embodiment, the clinical panel comprises a microarray chip with a plurality of oligonucleotides complimentary to human mtDNA with known or suspected mutations.

### Clinical Panels of the Invention

In one embodiment the clinical panel of the present invention comprises DNA probes complementary to nucleotide sequences of pathogens that cause human disease with similar symptoms. The exemplary but not limiting list of clinical panels for diagnosing human diseases that can be caused by a variety of pathogens is provided in Table 2.

TABLE 2
Clinical Panels

| | Clinical | |
|---|---|---|
| Panel/Disease | Sample | Pathogens/target moieties |
| Meningitis, | CSF | H. influenzae, N. meningitides; S. pneumoniae; S. |
| Primary | | pyogenes; E. coli; S. agalactiae; M. tuberculosis; L. |
| | | monocytogenes; C. neoformans; Enterovirus; Herpes |
| | | virus; Adenovirus; Arbovirus; Mumps virus |
| Meningitis, | CSF | C. albicans; Norcardia; T. pallidum; Borrelia; |
| Secondary | | Leptospira; H. capsulatum; C. immitis; N. fowleri; |
| | | Acanthamoeba; HIV, Cytomegalovirus |
| Food | Stool | Rotavirus, Astrovirus, Corona virus; E. coli O157; S. |
| poisoning or | | flexneri; S. sonnei; S. dysenteriae; S. boydii; S. typhi; S. |
| diarrhea | | enteridis; S. typhimurium; Y. enterocolitica; V. |
| | | cholerae; L. monocytogenes; S. aureus; C. difficile; B. |
| | | cereus; Y. pseudotuberculosis; C. jejunii; Giardia |
| | | lamblia |
| Sepsis | Blood | Coagulase negative Staphylococci; S. aureus; S. |
| | | pneumoniae; Enterococci; Pseudomonas; H. influenzae; |
| | | B. fragilis; C. albicans; S. enteritidis; S. typhimurium; |
| | | S. chloraesuis; E. coli; Citrobacter, Enterbacter; |
| | | Klebsiella; S. marcescens; S. mitis; S. sanguis; S. |
| | | mutans; S. salivarius; S. bovis; S. adjacens; S. |
| | | defectives; S. intermedius; S. anginosus; G. |
| | | morbillorum; S. mitior; S. agalactiae; H. aphrophilus; |
| | | A. actinomycetem comitans; C. hominis; Kingella; E. |
| | | corrodens |
| Acute | Sputum | S. pyogenes; C. pneumoniae; N. gonorrhoeae; C. |
| respiratory | | diphtheriae; M. pneumonia; A. haemolyticum; Candida; |
| infection | | Rhinovirus; Corona virus; Herpes simplex virus; |
| | | Epstein-Barr virus; Adenovirus; Coxsakievirus A; |
| | | Parainfluenza virus; Influenza virus |
| Chronic | Sputum, | S. aureus; S. pneumoniae; H. influenzae; M. catarrhalis; |
| respiratory | hemoptysis | K. pneumoniae; E. coli; P. aeruginosa; Legionella; M. |
| infection | | tuberculosis; Norcardia; M. pneumoniae; C. |
| | | pneumoniae; B. pertussis; C. trachomatis; C. psittaci; P. |
| | | carinii; Respiratory syncytial virus; Parainfluenza virus; |
| | | Rhinovirus; Adenovirus; Coxsakievirus A |
| UTI | urine | E. coli; P. mirabilis; Pseudomonas; Kebsiella; |
| | | Enterobacter; Enterococci; S. aureus; S. saprophyticus; |
| | | M. tuberculosis; Candida; Corynebacterium group D2; |
| | | U. urealyticum; M. pneumoniae; Adenovirus. |
| Blood | Donor blood | A, B, O, cis AB, Rh D, Rh E, Rh e, Rh C, Rh c human |
| transfusion | | genes; Hepatitis B virus; Hepatitis C virus; HIV; HTLV; |
| | | T. pallidum; Malaria |
| Rash | Blood, skin | C. psittaci; M. pneumoniae; R. rickettsii; R. akari; R. |
| | | prowazekii; R. typhi; R. (Orientia) tsutsugamushi; S. |
| | | typhi; F. tularensis; S. mobiliformis; T. pallidum; N. |
| | | gonorrhoeae; N. meningitidis; Leptospira; L. |
| | | monocytosgens; B. burgdorferi; P. aeruginosa; S. |
| | | aureus; S. pyogenes; C. canimorus; V. vulnificus; |
| | | Candida; C. neoformans; H. capsulatum; B. |
| | | dermatitidis; C. immitis; P. falciparum; HIV; Echo |
| | | virus; Coxackievirus; Measles; Adenovirus; |
| | | Lymphocytic choriomeningitis virus; Dengue virus; |
| | | Rubella; Yellow fever; Varicella-Zoster; Herpes |
| | | simplex; Varicella (Chickenpox); Cytomegalovirus; |
| | | Epstein-Barr virus; Hepatitis B virus; Parvovirus B19; |
| | | Human herpes virus 6 |
| STD | Urethral | C. trachomatis; N. gonorrhoeae; Mycoplasma; |
| | discharge, | Ureaplasma; T. pallidum; H. ducreyi; Candida; T. |
| | vaginal swab | vaginalis; Herpes simplex virus; Herpes virus; |
| | | Papilloma virus |
| Ear | Ear discharge | S. pneumoniae; H. influenzae; M. catarrhalis; P. |
| | | aeruginosa; Aspergillus; Candida; Respiratory syncytial |
| | | virus; Influenza virus; Enterovirus; Rhinovirus |
| Eye | Eye discharge | S. pneumoniae; S. aureus; Staphylococcus; H. |
| | | influenzae; Pseudomonas; M. catarrhalis; C. |
| | | trachomatis; N. gonorrhoeae; Acanthamoeba; |
| | | Adenovirus; Cytomegalovirus |
| Fever of | Blood, exudate | Any pathogens listed above |
| unknown | | |
| origin | | |

In one embodiment the clinical panel of the present invention comprises DNA probes complementary to nucleotide sequences of pathogens that cause symptoms associated with meningitis.

In another embodiment the clinical panel of the present invention comprises DNA probes complementary to nucleotide sequences of pathogens that cause symptoms associated with sepsis.

In another embodiment the clinical panel of the present invention comprises DNA probes complementary to nucleotide sequences of pathogens that cause symptoms associated with acute respiratory infection.

In another embodiment the clinical panel of the present invention comprises DNA probes complementary to nucleotide sequences of pathogens that cause symptoms associated with chronic respiratory infection.

In another embodiment the clinical panel of the present invention comprises DNA probes complementary to nucleotide sequences of pathogens that cause symptoms associated with urinary tract infection.

In another embodiment the clinical panel of the present invention comprises DNA probes complementary to nucleotide sequences of pathogens that cause symptoms associated with dermatitis (rash).

In another embodiment the clinical panel of the present invention comprises DNA probes complementary to nucleotide sequences of pathogens that cause symptoms associated with sexually transmitted diseases.

In another embodiment the clinical panel of the present invention comprises DNA probes complementary to nucleotide sequences of pathogens that cause symptoms associated with ear infections.

In another embodiment the clinical panel of the present invention comprises DNA probes complementary to nucleotide sequences of pathogens that cause symptoms associated with eye infections.

In another embodiment the clinical panel of the present invention comprises DNA probes complementary to nucleotide sequences of pathogens that cause symptoms associated with fever of unknown origin.

In another embodiment the clinical panel of the present invention comprises DNA probes complementary to nucleotide sequences of infectious agents and primary human blood groups.

In another embodiment the clinical panel of the present invention comprises DNA probes complementary to human mitochondrial DNA containing known or suspected mutations. Such mutations, and the diseases associated with the mutations are presented but not limited to mutations and diseases listed in Table 3.

TABLE 3
Mitochondrial mutation panels

| 1. | Mitochondrial mutation panel for LHON | |
|---|---|---|
| | (Leber hereditary optic neuropathy) | |
| | 1) | Primary mutations |
| | | MTND1 LHON 3460 G-A; |
| | | MTND1 LHON 4160 T-C; |
| | | MTND1 LHON 4171 C-A; |
| | | MTND2 LHON 5244 G-A; |
| | | MTATP6 LHON 9101 T-C; |
| | | MTCO3 LHON 9804 G-A; |
| | | MTND4L LHON 10663T-C; |
| | | MTND4 LHON 11778 G-A; |
| | | MTND5 LHON 13730 G-A; |
| | | MTND6 LDYT 14459 G-A; |
| | | MTND6 LHON 14482 C-G; |
| | | MTND6 LHON 14482 C-A; |
| | | MTND6 LHON 14484 T-C. |
| | 2) | Secondary mutations |
| | | MTND1 LHON 3394 T-C; |
| | | MTND1 LHON 4216 T-C; |
| | | MTND2 LHON 4917 A-G; |
| | | MTCO1 LHON 7444 G-A; |
| | | MTCO3 LHON 9438 G-A; |
| | | MTND5 LHON 13708 G-A; |
| | | MTCYB LHON 15257 G-A; |
| | | MTCYB LHON 15812 G-A. |
| | 3) | Miscellaneous |
| | | MTND1 LHON 3496 G-T; |
| | | MTND1 LHON 3497 C-T; |
| | | MTND1 LHON 3635 G-A; |
| | | MTND1 LHON 4136 A-G; |
| | | MTND2 LHON 4640 C-A; |
| | | MTCO3 LHON 9738 G-T; |
| | | MTND5 LHON-like 13528 A-G; |
| | | MTND6 LHON 14568 C-T. |
| 2. | Mitochondrial mutation panel | |
| | for Mitochondrial Encephalomyopathy. | |
| | 1) | Primary mutations |
| | | MTRNR1 DEAF 1555G; |
| | | MTTL1 MELAS 3243G (DM/DMDF, CPEO); |
| | | MTTL1 MELAS 3256T; |
| | | MTTL1 MMC 3260G; |
| | | MTTL1 MELAS 3271C; |
| | | MTTL1 MM 3302G; |
| | | MTT1 MMC 3303T; |
| | | MTTQ ADPD 4336C; |
| | | MTTS1 SNHL 7445G; |
| | | MTTS1 PEM/AMDF 7472insC; |
| | | MTTK MERRF 8344G; |
| | | MTTK MERRF 8356C; |
| | | MTATP6 NARP 8993 T-G; |
| | | MTATP6 NARP/Leigh Disease 8993 T-C; |
| | | MTATP6 FBSN/Leigh Disease 9176 T-C; |
| | | MTND5 MELAS 13513 G-A; |
| | | MMTTE MM + DM 14709C. |
| | 2) | Secondary panel |
| | | MTTF MELAS 583A; |
| | | MTTF Myoglobinuria 606G; |
| | | MTTF Tubulointerstitial nephritis 608G; |
| | | MTTF MM 618C; |
| | | MTRNR1 SNHL 1095C; |
| | | MTRNR1 DM 1310T; |
| | | MTRNR1 DM 1438G; |
| | | MTTV AMDF 1606A; |
| | | MTTV MELAS 1642A; |
| | | MTRNR2 Rett Syndrome 2835T; |
| | | MTRNR2 MELAS 3093G; |
| | | MTRNR2 ADPD 3196A; |
| | | MTTL1 KSS 3249A; |
| | | MTTL1 MM/CPEO 3250C; |
| | | MTTL1 MELAS 3252G; |
| | | MTTL1 MM 3254G; |
| | | MTTL1 DM 3264C; |
| | | MTTL1 PEM 3271delT; |
| | | MTTL1 DM 3271C; |
| | | MTTL1 Ocular myopathy 3273C; |
| | | MTTL1 LHON 3275A; |
| | | MTTL1 Myopathy 3288G; |
| | | MTTL1 MELAS 3291C; |
| | | MTND1 MELAS 3308 T-C; |
| | | MTNP1 NIDDM; LHON; PEO 3316 G-A; |
| | | MTND1 ADPD 3397 A-G; |
| | | MTT1 FICP 4269G; |
| | | MTT1 CPEO 4274C; |
| | | MTTI CPEO 4285C; |
| | | MTTI MHCM 4295G; |
| | | MTTI CPEO/MS 4298A; |
| | | MTTI MICM 4300G; |
| | | MTTI CPEO 4309A; |
| | | MTTI FICP 4317G; |
| | | MTTI Mitochondrial Encephalocardiomyopathy 4320T; |
| | | MTTQ Myopathy 4370insA; |
| | | MTTM MM 4409C; |
| | | MTTM Myopathy 4450A; |
| | | MTND2 AD 5460 G-A; |
| | | MTND2 AD 5460 G-T; |
| | | MTTW MM 5521A; |
| | | MTTW MILS 5537insT; |
| | | MTTW DEMCHO 5549A; |
| | | MTTA CPEO 5628C; |
| | | MTTN CPEO 5692C; |
| | | MTTN CPEO; MM 5703A; |
| | | MTTC Mitochondrial Encephalopathy 5814C; |
| | | MTTY Exercise Intolerance 5874G; |
| | | MTCO1 Myoglobinuria; Exercise Intolerance 5920 G-A; |
| | | MTCO1 Multisystem Disorder 6930 G-A; |
| | | MTTS1 MM 7497A; |
| | | MTTS1 SNHL 7510C; |
| | | MTTS1 SNHL 7511C; |
| | | MTTS1 PEM/MERME 7512C; |
| | | MTTD EEPR 7543G; |
| | | MTCO2 Mitochondrial Encephalomyopathy 7587T-C; |
| | | MTCO2 MM 7671 T-A; |
| | | MTCO2 Multisystem Disorder 7896 G-A; |
| | | MTCO2 Lactic Acidosis 8042 AT-del; |
| | | MTTK DMDF/MERRF 8296G; |
| | | MTTK MNGIE 8313A; |
| | | MTTK Mitochondrial Encephalopathy 8328A; |
| | | MTTK PEO and Myoclonus 8342A; |
| | | MTTK Cardiomyopathy 8348G; |
| | | MTTK MICM + DEAF/MERRF 8363A; |
| | | MTATP6 Leigh disease 9176 T-G; |
| | | MTCO3 Leigh-like 9537 C-CC; |
| | | MTCO3 Mitochondrial Encephalopathy 9952 G-A; |
| | | MTTG MHCM 9997C; |
| | | MTTG CIPO 10006G; |
| | | MTTG PEM 10010C; |
| | | MTTG GER/SIDS 10044G; |
| | | MTCO3 PEM; MELAS 9957 T-C; |
| | | MTND3 ESOC 10191 T-C; |
| | | MTND4 MELAS 11084 A-G; |
| | | MTND4 Exercise Intolerance 11832 G-A; |
| | | MTND4 DM 12026 A-G; |
| | | MTTH MICM 12192A; |
| | | MTTS2 CIPO 12246G; |
| | | MTTS2 DMDF 12258A; |
| | | MTTL2 CPEO 12308G; |
| | | MTTL2 CPEO 12311C; |
| | | MTTL2 CPEO 12315A; |
| | | MTTL2 MM 12320G; |
| | | MTND5 MELAS 13514 A-G; |
| | | MTND6 MELAS 14453 G-A; |
| | | MTCYB PD/MELAS 14787 TTAA-del; |
| | | MTCYB MM 15059 G-A; |
| | | MTCYB Exercise Intolerance 15150 G-A; |
| | | MTCYB Exercise Intolerance 15197 T-C; |
| | | MTCYB Mitochondrial Encephalomyopathy 15242 G-A; |
| | | MTCYB Exercise Intolerance 15615 G-A; |
| | | MTCYB MM 15762 G-A; |
| | | MTTT MM 15915A; |
| | | MTTT LIMM 15923G; |
| | | MTTT LIMM 15924G; |
| | | MTTT MM 15940delT; |
| | | MTTP MM 15990T. |

### EXAMPLE 1

To diagnose the causative agent of a disease described above, DNA is extracted and purified from patient's clinical sample and PCR-amplified with primers comprising nucleotide sequences specific for the pathogens suspected to cause the symptoms of the disease. Amplified DNA thus obtained is applied to a DNA chip of a Clinical Panel of the present invention and hybridized with the probes of the DNA chip. The amplified sample DNA hybridized with the probe(s) is labeled with means for labeling and detected with, for example, a confocal laser scanner. Only the probe that has nucleotide sequence complementary to the specific causative pathogenic agent of the symptoms of the disease hybridizes with the sample DNA and is detected.

### CONCLUSION

Thus, it will be appreciated that the clinical panel of the present invention may be used as a diagnostic tool for identification of the pathogen causing a disease; for identification of the mitochondrial mutation causing a disease; for screening donor blood for blood groups and infectious agents; and for prenatal genetic screening.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments and are exemplary and are not intended as limitations on the scope of the invention. Changes therein and other uses will occur to those skilled in the art which are encompassed within the spirit of the invention and are defined by the scope of the claims.

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions indicates the exclusion of equivalents of the features shown and described or portions thereof. It is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A clinical panel for differential diagnosis of disease comprising:
1) a DNA chip with at least 2 probes that have nucleotide sequences complementary to nucleotide sequences of pathogens causing diseases with similar symptoms;
2) primers for amplifying DNA obtained from clinical samples by PCR; and,
3) means for labeling amplified DNA hybridized with the probes of said DNA chip

2. A method of differential diagnosis of the disease comprising:
1) amplifying DNA obtained from clinical samples by PCR with primers specific to pathogens suspected to cause the disease;
2) applying the amplified DNA to the DNA chip of the clinical panel of the invention;
3) hybridizing the amplified DNA with DNA probes of the clinical panel;
4) labeling the amplified DNA hybridized with the probe(s) with means for labeling of the Clinical panel; and,
5) detecting DNA bound on the surface of the DNA chip after labeling.
